(19) United States
(12) Patent Application Publication
(10) Pub. No.: US 2018/0153782 A1
(43) Pub. Date: Jun. 7, 2018

(54) DRY SHAMPOO COMPOSITION

(71) Applicant: Church & Dwight Co., Inc., Princeton, NJ (US)

(72) Inventor: Shirish Desale, Folkestone (GB)

(73) Assignee: Church & Dwight Co., Inc., Princeton, NJ (US)

(21) Appl. No.: 15/832,190

(22) Filed: Dec. 5, 2017

Related U.S. Application Data
(60) Provisional application No. 62/430,572, filed on Dec. 6, 2016.

(51) Int. Cl. A61K 8/31; A61K 8/04; A61K 8/73; A61Q 5/02; A61Q 5/12

(52) U.S. Cl. CPC A61K 8/31; A61K 8/046; A45D 33/00; A61Q 5/02; A61Q 5/12; A61K 8/732

(57) ABSTRACT

The present disclosure provides dry shampoo compositions effective for cleaning hair and also moisturizing hair. The dry shampoo compositions can comprise a starch component, a conditioning agent, and optional further ingredients, such as propellants and fragrances.

### CROSS-REFERENCE TO RELATED APPLICATIONS

This application claims priority to U.S. Provisional Application No. 62/430,572, filed Dec. 6, 2016, which is hereby incorporated in its entirety by reference in this application.

### FIELD OF THE INVENTION

The present invention relates to compositions for cleaning hair, and more particularly to a dry shampoo composition.

### BACKGROUND

This invention relates to a natural, dry shampoo and conditioning composition capable of imparting superior cleaning and conditioning ability on hair.

Dry hair cleaning compositions for the rapid cleansing of hair are well known. These known compositions can be used on dry hair in order to freshen up the hair and/or to remove excess oils and other undesirables found on dirty hair. These known compositions are particularly useful when time is short and one cannot wash the hair using wet shampoo. Although these known compositions contain chemicals especially designed to cleanse the hair effectively without the use of water, many of these conventional cleaning compositions which are effective at cleansing dry hair, do not include a conditioning ability.

There are several 2-in-1 (i.e., cleaning and conditioning) dry shampoo products available on the market. A variety of conditioning materials are used in these known 2-in-1 products (e.g., cyclic silicones, petroleum based cationic surfactant, etc.). However, the conditioning materials used in conventional 2-in-1 dry shampoo products can be greasy and oily and can compromise the cleaning ability of the product.

Accordingly, there is still a desire and a need to provide a dry shampoo composition that is suitable for both cleaning dry hair as well as conditioning the dry hair without compromising the cleaning capability of the composition.

### SUMMARY OF THE INVENTION

In one aspect of the present invention, a dry shampoo composition is provided, the dry shampoo composition comprising a starch material, a hydrophobic emollient, particularly a long chain alkane, and optionally a propellant. The dry shampoo composition can further comprise a fragrance, for example. In some embodiments, the dry shampoo is substantially free of silicones. In various embodiments, the dry shampoo is substantially free of petroleum based cationic surfactants. In certain embodiments, the dry shampoo is substantially free of distearyldimonium chloride.

In various embodiments of the present invention, the emollient of the 2-in-1 dry shampoo plus conditioner composition comprises a C₁₃-C₁₅ alkane. In some embodiments, the emollient comprises hemisqualane. The starch material of the dry shampoo composition can comprise a rice starch, for example.

In certain embodiments of the present invention, the dry shampoo composition can comprise at least 0.1 weight percent of the starch material, based on the total weight of the dry shampoo composition; at least 0.1 weight percent of the emollient, based on the total weight of the dry shampoo composition; and at least 50 weight percent of the propellant, based on the total weight of the dry shampoo composition. In various embodiments, the dry shampoo composition can comprise about 1 to about 12 weight percent of the starch material, based on the total weight of the dry shampoo composition; about 1 to about 12 weight percent of the emollient, based on the total weight of the dry shampoo composition; and about 40 to about 95 weight percent of the propellant, based on the total weight of the dry shampoo composition.

In some aspects, the present invention also relates to a method of cleansing and conditioning hair. In various embodiments, the method can comprise applying a dry shampoo composition according to the present disclosure to the hair. In particular, the method can comprise spraying or sprinkling the dry shampoo composition on the hair and combing the dry shampoo composition through the hair. The dry shampoo composition can particularly be effective to improve conditioning of the hair. For example, conditioning of the hair can be improved such that the mean force required to comb through a tress of the hair after application of the dry shampoo composition is reduced by at least 20% relative to the mean force required to comb through the tress of the hair immediately prior to application of the dry shampoo composition. The improved conditioning effect particularly can be maintained for at least 8 hours, at least 12 hours, or at least 18 hours after application of the dry shampoo composition to the hair. Other aspects and advantages of the invention will be apparent from the following.

### BRIEF DESCRIPTION OF THE DRAWINGS

FIG. 1 is a plot of max load force (gf) grouped by time after treatment of hair with different dry shampoo compositions;

FIG. 2 is the Fourier-transform infrared spectroscopy (FTIR) spectra from virgin hair and sebum solution;

FIG. 3 is the FTIR spectra from virgin hair and two different dry shampoo composition products;

FIG. 4 is the FTIR spectra from virgin hair, hair after sebum application, and hair after 4 applications with a 2-in-1 dry shampoo composition product;

FIG. 5 is a bar graph showing values related to the FTIR image for the 1740 to 1635 cm⁻¹ intensity peak ratio;

FIG. 6 shows an IR image generated at regular spatial resolution to follow the product deposition on hair strands;

FIG. 7 shows an IR image generated at high spatial resolution to follow the product deposition on hair strands;

FIG. 8 shows an IR image generated at regular spatial resolution to investigate moisturizing effect for different dry shampoo composition products; and

FIG. 9 shows an IR image generated at high spatial resolution to investigate moisturizing effect for different dry shampoo composition products.

### DETAILED DESCRIPTION OF THE INVENTION

The present disclosure now will be described more fully hereinafter with reference to the accompanying drawings. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

In one aspect of the present invention, a dry shampoo composition is provided which can be topically applied to hair. The term “dry shampoo composition” is understood to relate to a product that is effective to cleanse hair without any necessity for rinsing with water following application. Wet shampoo compositions are understood to be applied to wet hair, lathered, and then rinsed away with added water. A dry shampoo composition, however, can be applied to hair and then optionally be brushed through the hair with the fingers or a utensil (e.g., a comb or brush) to effect the cleansing effect. No rinsing with water is required. A certain amount of liquid components may be included in a dry shampoo composition. Preferably, a dry shampoo composition as disclosed herein comprises less than 5 weight percent, less than 2 weight percent, less than 1 weight percent, or less than 0.1 weight percent of an aqueous component, particularly water, based on the total weight of the dry shampoo composition.

In certain embodiments, the dry shampoo composition can be sprinkled into the hair, or may be sprinkled into a user's hands and then applied to the hair. In various embodiments, the dry shampoo composition can be in the form of an aerosol which can be sprayed onto the hair of a user. The applied composition can be distributed through the hair by massaging into the hair, for example, in order to remove grease, oil and other undesirable elements from soiled hair. Any excess or residual dry shampoo composition powder can be removed from the hair by combing or brushing the hair, for example. In certain embodiments, the dry shampoo composition may leave little or no residue in the hair after application.

In various embodiments, the dry shampoo composition can include a plurality of components including, but not limited to, compounds for oil absorption and emollients. In some embodiments, the dry shampoo composition can further include additional components such as fragrances, propellants, natural oil (e.g., coconut oil or argan oil), synthetic oil, and the like. A dry shampoo composition of the present invention can comprise one or more of each type of component. However, each type of component is not necessarily present in different embodiments of the dry shampoo compositions described herein.

In various embodiments of the present invention, the dry shampoo composition can comprise a cleaning agent useful for removing (e.g., absorbing) oil, grease, and other undesirable elements from hair. For example, the dry shampoo composition can comprise at least one starch material that is effective to absorb at least a portion of any oils present on hair. Starch materials useful in the present invention include cornstarch, potato starch, tapioca starch, rice starch, wheat starch, cassaya starch, and combinations thereof. In certain embodiments, the dry shampoo composition comprises rice starch. A starch material can be modified (e.g., through processes such as esterification, etherification, oxidation, acid hydrolysis, crosslinking, or enzyme conversion) or unmodified.

In some embodiments of the present invention, the weight percentage of starch material in the dry shampoo composition, based on the total weight of the dry shampoo composition, can be about 0.1% to about 1.5%, about 1% to about 12%, about 2% to about 10%, or about 4% to about 8%. In certain embodiments, the weight percentage of starch material in the dry shampoo composition, based on the total weight of the dry shampoo composition, can be at least about 0.1%, at least about 1%, or at least about 4%, preferably with an upper range limit of about 50% by weight.

In various embodiments of the present invention, the dry shampoo composition can comprise an emollient useful for moisturizing the hair and thus acting as a conditioning agent. The emollient particularly is a hydrophobic emollient. For example, the emollient of the dry shampoo composition can comprise a long chain alkane. As used herein, a long chain alkane is at least a C₁₀ alkane or at least a C₁₂ alkane, preferably up to a C₄₀ alkane. For example, in certain embodiments the dry shampoo composition can comprise an emollient in the form of a C₁₃-C₁₅ alkane. In some embodiments, the emollient comprises a squalane and/or one or more derivatives thereof, particularly hemisqualane.

In some embodiments, the dry shampoo composition can comprise one or more additional conditioning agents known in the art, in addition to one or more hydrophobic emollients. Additional conditioning agents known in the art include, but are not limited to, silicones (e.g., phenyl trimethicones, dimethicones, cyclomethicones, dimethicone copolyols, amino silicones, etc.), petroleum based cationic surfactants, distearyldimonium chloride, guar compounds including cationic polymers and guar gum, polycationic compounds designated as polyquaternium 4, 6, 7, 10, or 22, etc. Some of the additional conditioning agents may provide other functions, such as being a solvent or a flow aid. Preferably, any such additional conditioning agents are present only in minor concentrations. In various embodiments of the present invention, additional conditioning agents can be expressly excluded from the dry shampoo composition such that one or more hydrophobic emollients (e.g., one or more long chain alkanes) are the only conditioning agents present in the dry shampoo composition. For example, in certain embodiments, the only conditioning agent present in the dry shampoo composition is a hydrophobic emollient such as hemisqualane. In some embodiments, the dry shampoo composition can be substantially free of additional conditioning agents beyond at least one hydrophobic emollient as described herein. As used herein, the term “substantially free of” means that the specified component is present in an amount of less than 0.1 percent by weight, based on the total weight of the composition. In some embodiments, the dry shampoo composition can be entirely free of additional conditioning agents, meaning that not even trace amounts of additional conditioning agents are present in the dry shampoo composition beyond the one or more hydrophobic emollients described herein—e.g., long chain alkanes. In certain embodiments, the dry shampoo composition is substantially or entirely free of silicones and/or petroleum based cationic surfactants. In some embodiments, the dry shampoo composition is substantially or entirely free of distearyldimonium chloride, for example.

In some embodiments of the present invention, the weight percentage of the emollients) in the dry shampoo composition, based on the total weight of the dry shampoo composition, can be 0.1% to about 15%, about 1% to about 12%, about 2% to about 10%, or about 4% to about 8%. In certain embodiments, the weight percentage of emollient(s) in the dry shampoo composition, based on the total weight of the dry shampoo composition, can be at least about 0.1%, at least about 1%, or at least about 4%, preferably with an upper limit of about 50% by weight.

In various embodiments of the present invention, the dry shampoo composition can comprise at least one fragrance. In some embodiments, a fragrance can be provided in the form of an essential oil. In certain embodiments, the weight percentage of one or more fragrances in the dry shampoo composition, based on the total weight of the dry shampoo composition, can be about 0.01% to about 3%, about 0.1% to about 2%, or about 0.1% to about 1%. In certain embodiments, the weight percentage of a fragrance in the dry shampoo composition, based on the total weight of the dry shampoo composition, can be at least about 0.01%, at least about 0.1%, or at least about 1%, preferably with an upper limit of about 10% by weight.

In some embodiments, a dry shampoo composition is provided as an aerosol (e.g., an aerosol dry shampoo composition) and can comprise at least one propellant. Non-limiting examples of propellants include butane, isobutane, propane, liquefied petroleum gas, dimethyl ether, trichlorofluoromethane, dichlorodifluoromethane, dichlorotetrafluorothane, monochlorodifluoromethane, trichlorotrifluoroethane propane, carbon dioxide, nitrous oxide, and combinations thereof. The term “aerosol dry shampoo composition,” as used herein, refers to a composition comprising a dry shampoo composition and a propellant. When a dry shampoo composition is provided as an aerosol as compared to a powder, it may advantageously allow for the dry shampoo composition to be applied in a diffusive manner, and may increase the transparency of the dry shampoo composition when compared to powder application. After spraying the aerosol dry shampoo composition onto the hair, the propellant in the dry shampoo composition evaporates and a dry powder remains.

In some embodiments of the present invention, the weight percentage of the propellant, in the dry shampoo composition, based on the total weight of the dry shampoo composition, can be about 20% to about 98%, about 40% to about 95%, or about 76% to about 90%. In certain embodiments, the weight percentage of carrier material in the dry shampoo composition, based on the total weight of the dry shampoo composition, can be at least about 20%, at least about 40%, at least about 60%, or at least about 80%, preferably with an upper limit of about 98% by weight.

The present dry shampoo composition not only provides a cleansing effect but also provides conditioning to the treated hair. Conditioning can separately mean providing a moisturizing effect to the hair or providing softness and smoothness to the hair. Preferably, the present dry shampoo composition provides all of softness, smoothness, and moisturizing.

In some embodiments, conditioning of the hair can be defined in relation to mean force applied in a combability test, as described in Example 1 and Example 2. Preferably, the present dry shampoo composition provides conditioning for at least 8 hours, at least 12 hours, or at least 18 hours after application of the dry shampoo composition to hair. Conditioning is defined as the mean used to comb through a tress of the treated hair being less than the mean force used to comb through the tress immediately prior to treatment. The reduction in mean force for the treated tress of hair is at least a 10% reduction, at least a 20% reduction, at least a 30% reduction, or at least a 40% reduction (with a maximum of 100%, 150%, or 200% reduction). The noted percent reduction can be present at any of the noted times post application of the dry shampoo composition to the hair. In particular, there can be a 10% to 100% reduction, a 15% to 90% reduction, or a 20% to 80% reduction in combability mean force at one or more of 8 hours, 12 hours, and 18 hours post application of the dry shampoo composition to a tress of hair. As such, the present product can be characterized as being up to an 18 hour conditioning composition.

A method of preparing a dry shampoo product is also provided herein. In some embodiments, a method of preparing a dry shampoo product can comprise providing a delivery container, adding an oil absorbing agent to the delivery container, adding an emollient to the container (separately or in combination with a fragrance), and adding a propellant to the delivery container. In further embodiments, the method of preparing a dry shampoo product can comprise providing a delivery container, adding an emollient to the container (separately or in combination with a fragrance), adding an oil absorbing agent to the delivery container, and adding a propellant to the delivery container.

### EXPERIMENTAL

### Example 1

The performance of a 2-in-1 dry shampoo according to the present disclosure was evaluated. The composition is provided in Table 1 (with weight percentage ranges being based on the total weight of the respective dry shampoo composition).

TABLE 1
Composition of the 2-in-1 Dry Shampoo Conditioner of the
Present Disclosure

| Material | INCI Name | Weight Percentage Range |
|---|---|---|
| Propellant | Butane, Isobutane, Propane | 70.0%-90.0% |
| Starch | Oryza Sativa (Rice) Starch, | 4.0%-8.0% |
| | Cetrimonium Chloride | |
| Fragrance | Parfum | 0.1%-1.0% |
| Emollient | C13-C15 alkane | 4.0%-8.0% |

The performance of the Example 2-in-1 dry shampoo versus the performance of a control at 12 hours, 18 hours, and 24 hours was evaluated by measuring dry combing force using the Dry Combing Method described below. Caucasian medium brown hair tresses were used (supplied by International Hair Importers). Each tress was 8 inches long, 1 inch wide, and weighed approximately 3.0 g. Before treatment, tresses were bleached using standard bleaching procedure with 6% hydrogen peroxide solution at pH 10.2 and 40° C. for 40 min in total.

Tresses were standardized by treating all of the tresses with 10 wt. % sodium laureth sulfate (SLES), massaging 30 s, and rinsing for 30 s (40 C, 1 GPM flow rate). Treated tresses in Cell 2, Cell 3, and Cell 4 were left to equilibrate for 12, 18 and 24 hrs respectively at 21° C. and 60% RH prior to testing by Dry Combing. Preparation of the tresses in the four test cells (with 8 tresses per cell) is summarized below.

Cell 1: Control—10% SLES treatment only

Cell 2: 10% SLES treatment followed by spray application of Example 2-in-1 Shampoo plus Conditioner, testing carried out 12 hrs after the spray application

Cell 3: 10% SLES treatment followed by spray application of Example 2-in-1 Shampoo plus Conditioner, testing carried out 18 hrs after the spray application 18 hrs

Cell 4: 10% SLES treatment followed by spray application of Example 2-in-1 Shampoo plus Conditioner, testing carried out 24 hrs after the spray application

The primary technical function of most conditioning products is to lubricate the hair surface; and, in doing so, facilitate manageability and provide detangling benefits and lower combing friction. A common and highly consumer-relevant approach for measuring this lubrication involves an instrumental combing experiment. Testing involves use of an Instron tensile tester to measure frictional forces while a hair tress is pulled through a comb. Testing was performed in accordance with the widely-used method first proposed by Garcia & Diaz (JSCC, 27, (1976) 379-398—Combability Measurements on Hair). Combing experiments were performed in the dry state after treatment. Six combing strokes were performed per tress, while eight replicate hair tresses were used per sample to ensure statistical relevance.

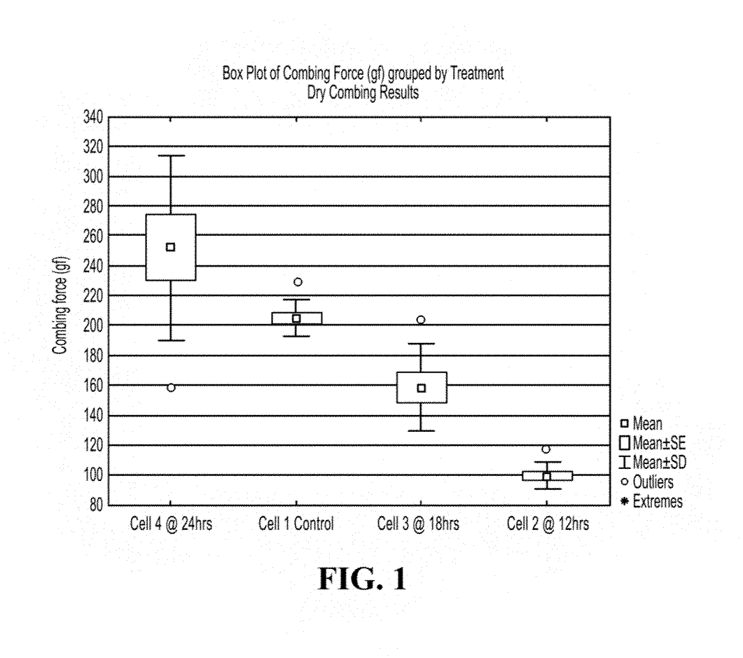

FIG. 1 shows a plot created using Statistica™ of the maximum load force (gf) vs. treatment. In addition, JMP™ analytical software was used to perform statistical analysis, which is shown below in Table 2. Statistics were performed using the student's t-test at the 95% confidence level.

TABLE 2
Statistical Analysis by JMP software. Treatments not connected with
the same letter are significantly different

| | | Mean | Std | Std Err | | | | |
|---|---|---|---|---|---|---|---|---|
| Treatment | N | Force | Dev | Mean | | | | |
| Cell 4 at 24 hrs | 8 | 252.39 | 62.03 | 21.93 | A | | | |
| Cell 1 Control | 8 | 204.85 | 12.17 | 4.30 | | B | | |
| Cell 3 at 18 hrs | 8 | 158.49 | 28.69 | 10.15 | | | C | |
| Cell 2 at 12 hrs | 8 | 99.06 | 8.97 | 3.17 | | | | D |

There is a statistically significant difference comparing Cell 3@ 18 hrs and Cell 2@ 12 hrs to the control Cell 1. Cell 4 @ 24 hrs shows a significantly higher combing force from Cell 1 Control, Cell 2 @ 12 hrs and Cell 3 @ 18 hrs. Cell 2 @ 12 hrs indicates the least combing force. A decrease in combing force of approximately 23% was measured for Cell 3 @ 18 hrs when compared to the control Cell 1. A decrease in combing force of approximately 52% was measured for Cell 2 @ 12 hrs when compared to the control Cell 1. The calculated percent value as detailed above is based solely on the average mean of all the data.

The above data illustrates the ability of the Example product to provide a cleansing as well as a conditioning effect. At 12 hours after application, the approximately 52% reduction in mean force (relative to control) shows a significant improvement in softness and smoothness of the hair because of the conditioning effect provided by the Example product. At 18 hours, the approximately 23% reduction in mean force (relative to control) illustrates the ability of the Example product to provide improved softness and smoothness for a time exceeding what would be expected by most consumers.

### Example 2

The efficiency of two dry hair products to clean hair tresses by removing sebum from hair fibers was evaluated. The products tested were 1) Commercial Dry Shampoo described in Table 3 below; and 2) Example 2-in-1 Shampoo plus Conditioner according to the present disclosure, the formula of which is provided above in Example 1.

TABLE 3
Composition of the Commercial Dry Shampoo

| Trade Name | INCI Name | Weight Percentage |
|---|---|---|
| Propellant | Butane, Isobutane, Propane | 70%-90% |
| Starch | Oryza Sativa (Rice) Starch, | 4.0%-8.0% |
| | Cetrimonium Chloride | |
| Solvent | Alcohol Denat. | 4.0%-8.0% |
| Fragrance | Parfum | 0.1%-1.0% |
| Flow Aid | Distearyldimonium Chloride | 0.001%-0.1% |

Caucasian medium brown hair tresses were used (supplied by International Hair Importers). Each tress was 8 inches long, 1 inch wide and weighed approximately 3.0 g. Synthetic sebum (available from Scientific Service S/D, Inc.) was applied in an amount of 0.5 g to virgin hair tresses to mimic unwashed hair. The tresses were combed for even distribution of the sebum along the hair fibers. The hair tresses were sprayed with one of the test products on both sides at 3 different locations (top, middle and bottom part of the hair tresses). After application hair tresses were massaged manually for 30 s. After 10 minutes the tresses were combed 10 times on each side. For each product, 4 applications were performed on the same hair tresses. At the end of the experiments hair tresses were washed with standard (SLES) treatment.

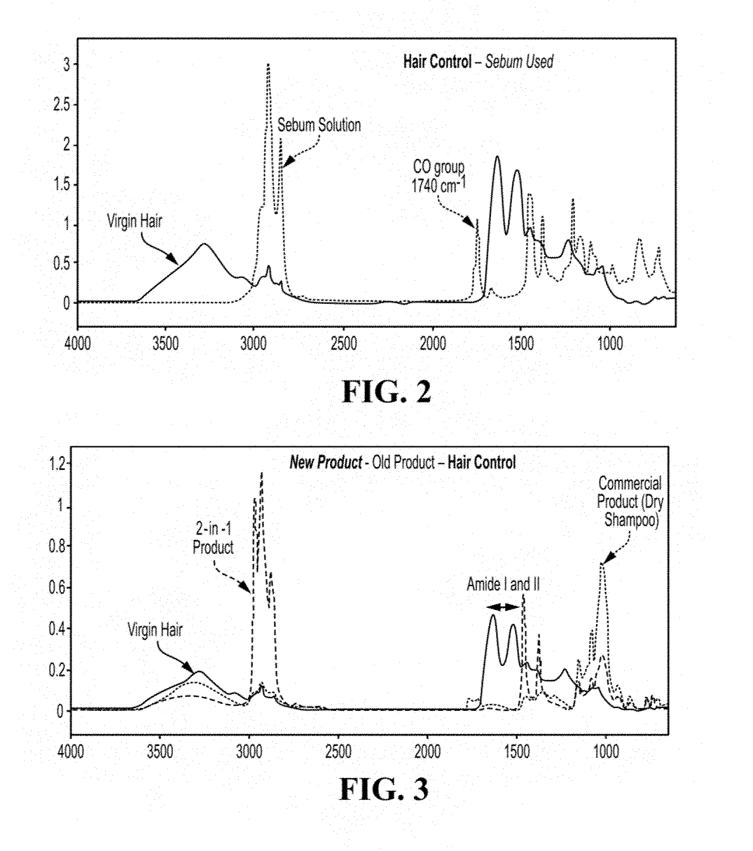

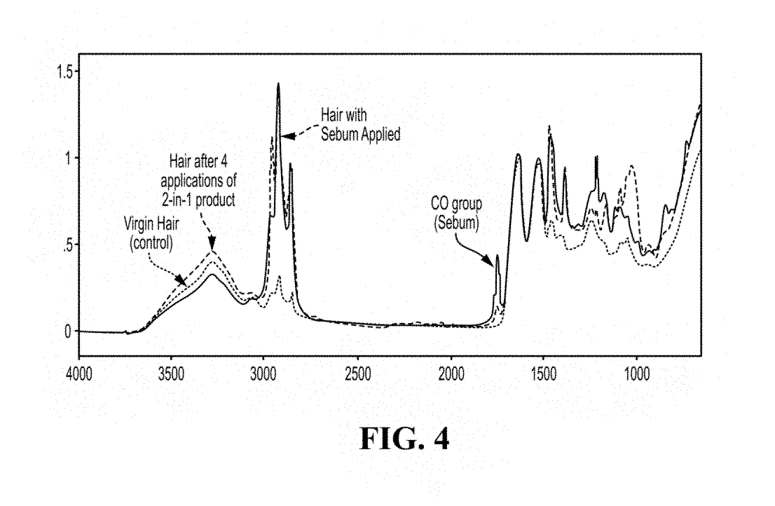

The Fourier-transform infrared spectroscopy (FTIR) data were generated with a spotlight system 400 from PerkinElmer with an ATR accessory. In the FTIR spectra, position and band intensity gave some information about the chemical nature of the material. For example, the contribution of esters always have a carbonyl (C═O) band around 1740 cm⁻¹. This band was used in this project to follow the presence and the reducing content of sebum on the hair tresses after dry shampoo application. FIG. 2 is an FTIR spectra from virgin hair with the applied sebum solution. For each product the hair samples were tested at the beginning as a negative control; after sebum application as a positive control; after 1 product application; after 2 product applications; after 4 product applications; and after regular wet shampoo wash. To be statistically significant and take into consideration the important variation inside the same hair tress, 9 FTIR spectra were recorded along various parts of the tress (3 root/3 middle/3 tip) for each measurement. For each condition, nine spectra were recorded, baseline corrected, and averaged. A CO (sebum)/protein (Amide I) ratio was defined and calculated to assess the amount of sebum on the hair fibers. FIG. 3 is an FTIR spectra from virgin hair, a commercial dry shampoo product, and an embodiment of the 2-in-1 product of the present invention. Indeed, as shown in FIG. 3, there was not contribution from the 2 products used in this study in the Amide I area so this band was used to normalize the sebum content. The carbonyl (C═O) band around 1740 cm⁻¹ was used to follow the presence of sebum on the hair fibers (see FIG. 4). FIG. 4 is the FTIR spectra from virgin hair, hair after sebum application, and hair after 4 applications with a 2-in-1 dry shampoo composition product of the present invention.

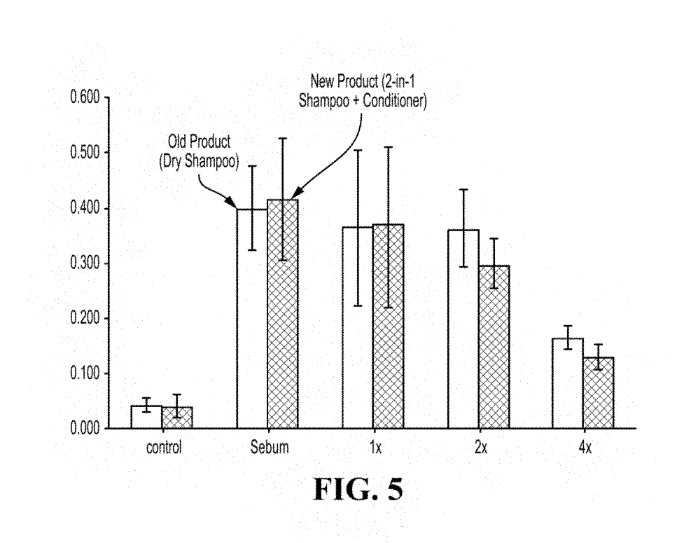

To visualize the presence of sebum on the hair fibers we generated an IR image by calculating the 1740 to 1635 cm⁻¹ intensity peak ratio. After the sebum application, the sebum content on the hair surface is extremely important compared to the control where almost no sebum was detected. After each product application, it was observed that the sebum content decreased, indicating that the dry products can remove sebum from the hair fibers. The new product 2-in-1 removed more sebum than the old product, especially after 2 and 4 applications. After 4 applications for both old and new products, the sebum content on the hair fibers is significantly different compared to the control. To recover the initial level observed for the sebum, a regular wet washing step was necessary. The results of the sebum removal are shown in FIG. 5, which shows values related to the FTIR image for the 1740 to 1635 cm⁻¹ intensity peak ratio. Commercial (“old”) dry shampoo product is the left bar for each sample, and an embodiment of the new 2-in-1 product of the present invention is represented by the right bar for each sample. The data used to create the bar graph of FIG. 5 is also presented in Table 4 below.

TABLE 4
Values related to the FTIR image for the 1740 to 1635 cm⁻¹ intensity
peak ratio

| | | | | |
|---|---|---|---|---|
| Control | Sebum | 1x | 2x | 3x |

AVERAGE

| | | | | | |
|---|---|---|---|---|---|
| Old Product | 0.042 | 0.398 | 0.362 | 0.360 | 0.164 |
| New Product | 0.041 | 0.413 | 0.362 | 0.296 | 0.130 |

STANDARD DEVIATION

| | | | | | |
|---|---|---|---|---|---|
| Old Product | 0.01 | 0.08 | 0.14 | 0.07 | 0.02 |
| New Product | 0.02 | 0.11 | 0.15 | 0.05 | 0.02 |

In summary, both products, the commercial dry shampoo and the Example 2-in-1 dry shampoo, are effective to clean hair tresses by removing sebum from the hair surface. The 2-in-1 removed more sebum than the old product, especially after several applications. After 4 applications, the sebum content on the hair fibers is significantly different when comparing the control with both products.

### Example 3

The potential moisturizing effect of two dry hair products along hair fibers using an ATR-FTIR imaging system is evaluated and compared. The products tested were 1) Control; 2) Commercial Dry Shampoo; and 3) Example 2-in-1 Shampoo plus Conditioner. The formulas of the test products 2 and 3 are the same as in Tables 1 and 3 above.

Caucasian medium brown hair tresses were used (supplied by International Hair Importers). Each tress was 8 inches long, 1 inch wide and weighed approximately 3.0 g. The tresses were standardized by washing step with 10% SLES. The hair tresses were sprayed with one of the dry products on both sides at 3 different locations (top, middle and bottom part of the hair tresses). After application, the hair tresses were massaged manually for 30 s. After 10 minutes, the tresses were combed 10 times on each side. A spotlight system 400 from Perkin Elmer was used to record all data.

For this method, single hair fibers were randomly selected and analyzed for each sample. ATR-FTIR spectroscopic images along hair fiber provided complete spatially resolved IR spectra of the hair surface with a spatial resolution up to 1.5 μm. This method, thanks to its high spatial resolution, can highlight some modifications in a specific sample area (cuticle area, outer layer of the cortex, etc.) which are weak and almost invisible with the regular FUR image. This method provided hyperspectral images, which were used to analyze the deposition and the distribution of hair dry products along hair fiber and to detect potential hair modifications such as moisturizing.

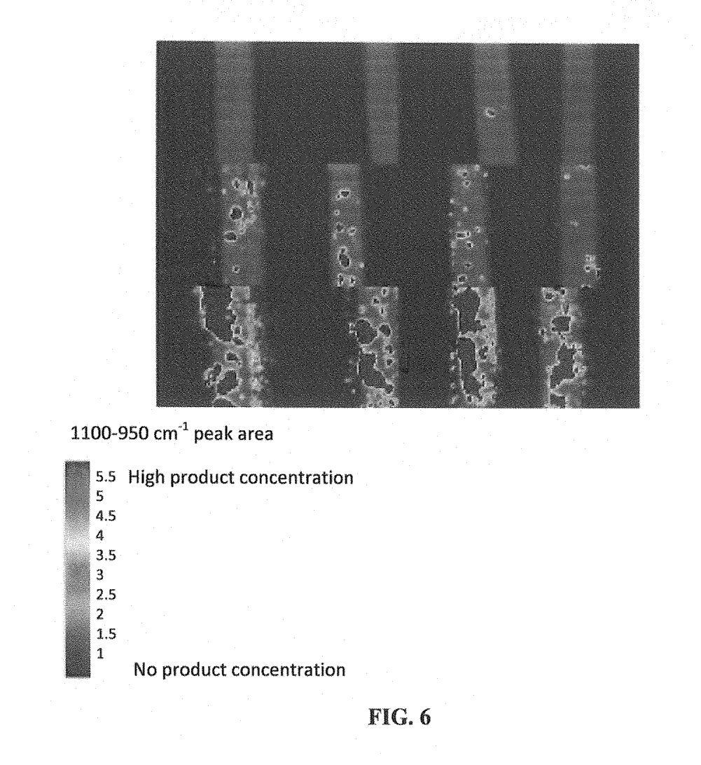

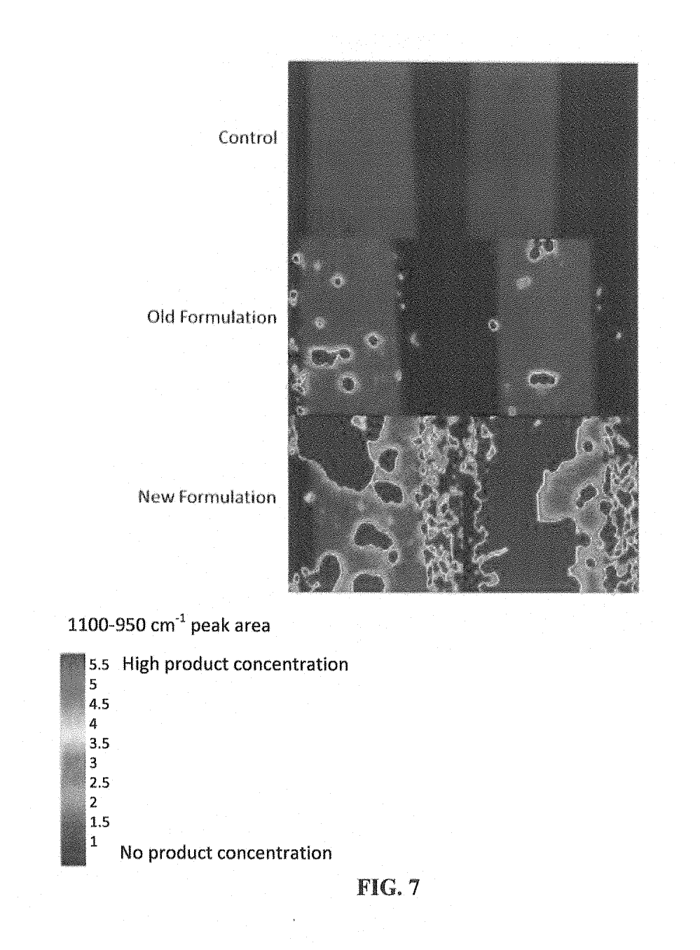

For each group (control, the commercial dry shampoo, and the Example 2-in-1 dry shampoo plus conditioner formulation), 6 fibers were scanned. Two fibers were analyzed at high resolution and 4 fibers at regular spatial resolution. The IR images generated by this system used false color, where highest intensity appears in red and lowest intensity appears in blue. FIG. 6 is a regular spatial resolution IR image generated to follow the product deposition. Both products presented an intense peak around 1030 cm⁻¹, which was used to generate this IR image. As can be observed, there is a significant difference between the two products used in this project. The 2-in-1 product shows clearly a more uniform and significant deposition along the hair fibers compared to the dry shampoo composition without an emollient. An IR image was also generated at high spatial resolution to follow the product deposition using the intense peak around 1030 cm⁻¹ to generate the IR image (see FIG. 7). As observed in the regular spatial resolution IR image, there is a significant difference between the two dry hair products. The 2-in-1 product clearly showed a higher deposition along the hair fibers and a more uniform distribution compared to the dry shampoo product without an emollient.

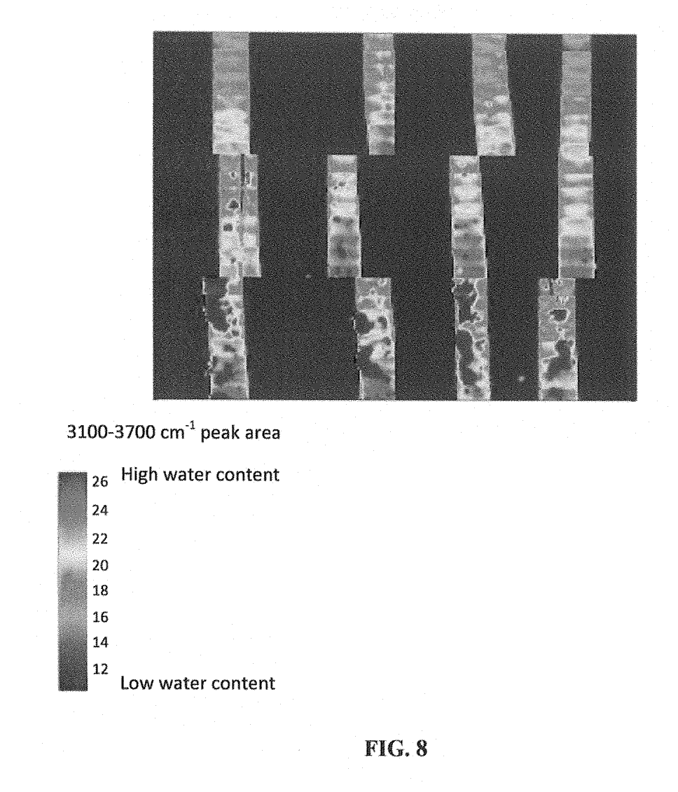

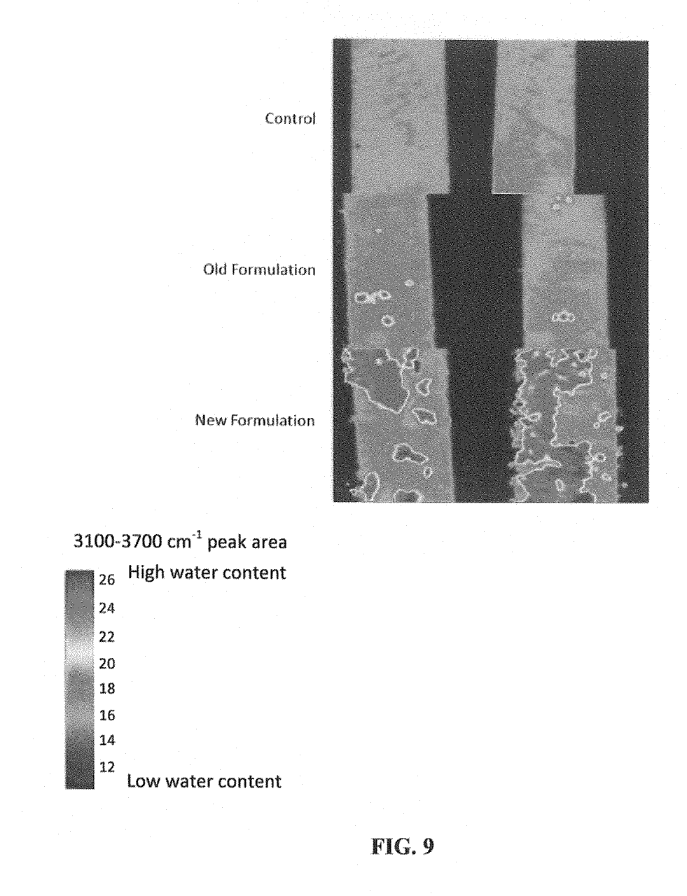

The same hyperspectral IR image was used to investigate the potential moisturizing effect. FIG. 8 shows an IR image that was generated using the 3700-3100 cm⁻¹ peak area. Clearly both products improved the water content at the hair surface as compared to the control sample. The 2-in-1 formulation seems more effective. The correlation between the product deposition and the water content is extremely strong. Without being limited by theory, the higher moisturizing effect observed with the 2-in-1 formulation could be a result of the higher deposition along the hair fibers detected with the 2-in-1 dry hair product. An IR image was also generated at high spatial resolution to follow the product deposition using the intense peak around 3700-3100 cm⁻¹ to generate the IR image (see FIG. 9). As observed in the regular spatial resolution IR image, there is a significant difference between the two dry hair products.

In summary, both products effectively improved water content at the hair surface. The active deposition observed after treatment with the 2-in-1 product is higher and more homogenous along the hair fibers as compared to treatment with the dry shampoo composition without an emollient. The 2-in-1 product is more effective in terms of moisturizing than the dry shampoo composition without an emollient.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A dry shampoo composition, comprising:
a starch material;
a hydrophobic emollient; and
optionally a propellant.

2. The dry shampoo composition of claim 1, wherein the dry shampoo is substantially free of silicones.

3. The dry shampoo composition of claim 1, wherein the dry shampoo is substantially free of petroleum based cationic surfactants.

4. The dry shampoo composition of claim 1, wherein the dry shampoo is substantially free of distearyldimonium chloride.

5. The dry shampoo composition of claim 1, wherein the hydrophobic emollient comprises a long chain alkane.

6. The dry shampoo composition of claim 1, wherein the hydrophobic emollient comprises a C₁₃-C₁₅ alkane.

7. The dry shampoo composition of claim 1, wherein the hydrophobic emollient comprises hemisqualane.

8. The dry shampoo composition of claim 1, wherein the starch material comprises a rice starch.

9. The dry shampoo composition of claim 1, comprising:
at least 0.1 weight percent of the starch material, based on the total weight of the dry shampoo composition;
at least 0.1 weight percent of the hydrophobic emollient, based on the total weight of the dry shampoo composition; and
at least 50 weight percent of the propellant, based on the total weight of the dry shampoo composition.

10. The dry shampoo composition of claim 1, comprising:
about 1 to about 12 weight percent of the starch material, based on the total weight of the dry shampoo composition;
about 1 to about 12 weight percent of the hydrophobic emollient, based on the total weight of the dry shampoo composition; and
about 40 to about 95 weight percent of the propellant, based on the total weight of the dry shampoo composition.

11. The dry shampoo composition of claim 1, further comprising a fragrance.

12. A method of cleansing and conditioning hair, the method comprising applying a dry shampoo composition according to claim 1 to the hair.

13. The method of claim 12, comprising spraying the dry shampoo composition on the hair and combing the dry shampoo composition through the hair.

14. The method of claim 12, wherein conditioning of the hair is improved such that the mean force required to comb through a tress of the hair after application of the dry shampoo composition is reduced by at least 20% relative to the mean force required to comb through the tress of the hair immediately prior to application of the dry shampoo composition.
